(19) United States
(12) Patent Application Publication
(10) Pub. No.: US 2020/0229804 A1
(43) Pub. Date: Jul. 23, 2020

(54) COUGH TREATMENT DEVICE

(71) Applicant: Min Ho CHANG, Jeonju-si (KR)

(72) Inventor: Min Ho CHANG, Jeonju-si (KR)

(21) Appl. No.: 16/651,095

(22) PCT Filed: Jul. 31, 2018
(86) PCT No.: PCT/KR2018/008686

§ 371 Date: Mar. 26, 2020

(30) Foreign Application Priority Data
Sep. 28, 2017 (KR) 10-2017-0125991

(51) Int. Cl. A61B 13/00; A61F 5/56

(52) U.S. Cl. CPC A61B 13/00; A61F 5/566

(57) ABSTRACT

The present invention relates to a cough treatment device, and in order to treat coughs, which are the main symptoms of a respiratory disease, provided is the cough treatment device having: a horizontal bar formed in a bar shape so as to press, while being bitten by the upper and lower teeth, the tip of the tongue such that the tongue enters the mouth, thereby expanding an airway; and wing parts for guiding air flowing into the mouth such that the air passes the surface of the mouth, thereby buffering air flowing into the airway and changing the temperature of the air and, simultaneously, allowing moisture in the air.

### TECHNICAL FIELD

The present invention relates to a cough treatment apparatus, more particularly, to a cough treatment device which includes a wing part for guiding air introduced into an oral cavity to an inner wall surface of the mouth at the end of a horizontal bar held between upper teeth and lower teeth, thereby changing a temperature of the air introduced into the respiratory tract and, simultaneously, supplying moisture to the air.

### BACKGROUND ART

In general, a cold is an infection of the upper respiratory tract system including nose and throat by viruses and germs, wherein symptoms may include sneezing, nasal congestion, runny nose, sore throat, cough, fever, headache and muscle aches.

In particular, when catching cold, inflammation and heat occur in the tonsil and larynx due to diverse causes such as infection. Due to this, the area around the respiratory tract becomes swollen and narrowed, and may show deformation in shape. As a result, sputum or saliva may enter the lungs and, especially, could be caught or gathered in the epiglottis valley between the tongue and the epiglottis so as to easily overflow, thus causing cough and pain.

Currently, a bronchodilator or a patch type product is commercially available, however, such a composition product is uncertain in terms of recovery effects and has a limitation in quick response.

Further, hacking cough may often stimulate inflammation, irritate a fever area and further cause neurological reaction, which is exacerbated into persistent spasmodic cough. If this continues for a long time, there is a risk of progressing to lung infection. However, it is difficult to treat coughing itself due to complex mechanisms by which coughing is induced.

In particular, due to fever and pain during coughing, breathing is reflectively performed through the mouth. This results in water evaporation inside the respiratory tract, thus drying the same. Herein, it is to be noted that water evaporation stimulates fever and moisture drying in an inflamed area and, when swallowing saliva, further irritates the respiratory tract including prickly pain as the respiratory tract is soaked by the saliva. This increases pain and further stimulates the nerves, thus causing more coughing. As a result, the fever and inflammation become worse and coughing continues. In order to solve the above problems, frequently drinking warm water has been recommended. However, this is a temporary method and still has a problem of persistent coughing.

Currently-used cough medicines are antitussive expectorants, which are usually anesthetics.

Codeine as a major component of Codenal Tab. (Sam-A Pharm. Co., Ltd), which is a central nervous system-active antitussive expectorant and a drug substance contained in a very small amount wherein its dosage is limited by age and caution should be taken as to side effects.

In addition, it is known that use of strong antitussive drugs may cause adverse effects and, for vulnerable groups such as pregnant women, children, cancer patients, etc. who feel burden with drug or medication, alternative cough treatments are urgently required.

Currently, snoring prevention devices have been disclosed as a means of resolving respiratory tract narrowing or symptoms due to airflow and, when the throat is swollen at the time of sleep, air flow through the respiratory tract is disturbed and the respiratory organ itself trembles, causing vibration noise, resulting in snoring.

Accordingly, the snoring prevention mechanism for injecting air into the respiratory tract according to patent document 1 (KR10-2015-0010450 A) includes a tongue-accommodating main body which is in close contact with a top surface of the tongue and encloses the tongue; and a spacing part which protrudes upward from the top surface of the tongue accommodating main body and is in close contact with the vault of the mouth (“palate”).

Herein, the tongue accommodating main body (“main body”) is formed in an arch shape and is in close contact with the top surface of the tongue, wherein the main body presses the tongue downward in such a state as described above.

Further, a portion of the spacing part contacting the palate presses the main body while being in close contact with the palate.

As a result, a space is formed between the palate and the main body, and the air flowing into the mouth through the space speedily passes through the respiratory tract to enable smooth respiration of a wearer of the device.

However, in the above described patent document 1 (KR10-2015-0010450 A), when there is inflammation and heat in the respiratory tract external air passes directly through the space between the palate and the main body, drying the respiratory tract and, especially, air having high temperature difference and high pressure air may rub against the respiratory tract and cause coughing. This worsens inflammation, fever, and swelling of the respiratory tract, narrowing the same. Accordingly, if there is inflammation and heat in the respiratory tract, the above device in the prior art could not be used in both snoring and coughing treatments.

### DISCLOSURE

### Technical Problem

The present disclosure has been proposed to overcome the aforementioned problems, and an object of the present disclosure is to provide a cough treatment device, including: a horizontal bar which is formed in a bar shape, and may press the end of the tongue to enter the oral cavity while being held between upper and lower teeth, thereby extending the respiratory tract; and a wing part which guides air flowing into the mouth to pass through the inner wall surface of the mouth, so as to change a temperature of the air introduced into the respiratory tract while enabling the air to contain moisture, simultaneously.

### Technical Solution

In order to achieve the purposes of the present invention, the cough treatment device according to the present invention may include: a horizontal bar which is formed in a non-penetrating bar shape to press the end of the tongue in order to introduce the tongue into the oral cavity, wherein top and bottom surfaces of the bar are held between the upper and lower teeth and are exposed to the outside, and the horizontal bar may guide the air introduced into the mouth through a gap between the upper and lower teeth to flow in a horizontal direction; and a wing part which protrudes from both sides of the horizontal bar at the end thereof and induces the air guided by the horizontal bar and introduced to the oral cavity to be diverted toward a wall surface of the oral cavity and flow into the respiratory tract.

With respect to the cough treatment device according to the present invention, the horizontal bar may include: a lower bar having the wing part formed at the end thereof; and an upper bar which protrudes from a top surface of the lower bar in a bar shape and has a thru-hole formed on both sides at the end of the upper bar in order to pass air introduced into the oral cavity in a horizontal direction.

With respect to the cough treatment device according to the present invention, the wing part may be formed in any one of semicircular, semi-elliptic and polygonal shapes.

With respect to the cough treatment device according to the present invention, the wing part may further include at least one passage groove formed on an outer surface thereof.

### Advantageous Effects

According to the present invention, firstly, when the throat is continuously open through respiratory tract extension, the breathing becomes active so as to effectively reduce heat at the inflammation site.

Second, when the tongue is pressed backward by the horizontal bar, a space in the epiglottis valley between the tongue and the epiglottis, as a main cause of respiratory tract obstruction, may be narrowed, thus preventing the secretion from being caught or gathered and then overflowing.

Third, an angle of the lower jaw opened by the horizontal bar and the backward pressing of the tongue may temporarily block the secretion generated in the oral cavity so that the secretion does not flow in the respiratory tract direction but is gathered in the oral cavity, thereby relieving coughing caused by the secretion.

Fourth, if the inflow air is diverted toward the wall surface by the wing part, it is possible to maintain stable breathing by eliminating coughing factors such as dry throat due to air, high difference in temperature, high air friction, etc.

Fifth, a temperature of respiratory air at room temperature of about 25° C. may be elevated and maintained at an average of about 32° C., and a surface temperature of the oral cavity may be gradually decreased from the body temperature to about 34° C. to thus relieve the fever, but is not too low, thereby achieving effects of treating cough and preventing recurrence thereof.

### DESCRIPTION OF DRAWINGS

FIG. 1 is a perspective view of the cough treatment device according to the present invention.

FIG. 2 is a plan view of the cough treatment device according to the present invention.

FIG. 3 is a side view illustrating the cough treatment device fixed by holding the same in the mouth.

FIG. 4 illustrates another embodiment of the wing part in the cough treatment device according to the present invention.

### BEST MODEL

Hereinafter, preferred embodiments of the present invention will be described in detail with reference to the accompanying drawings.

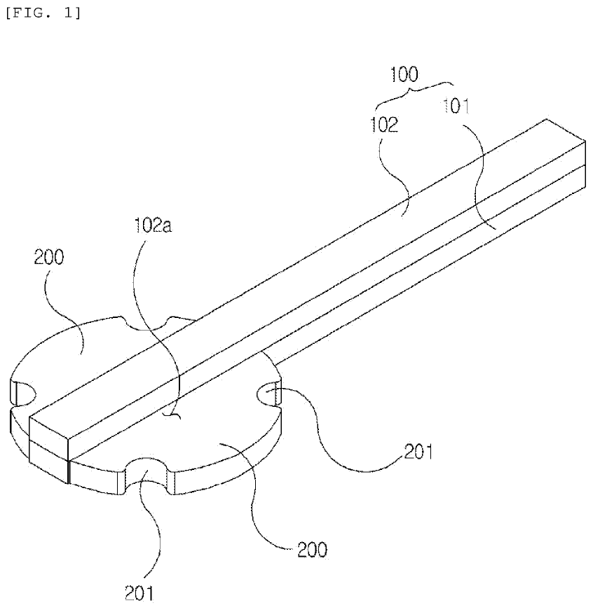

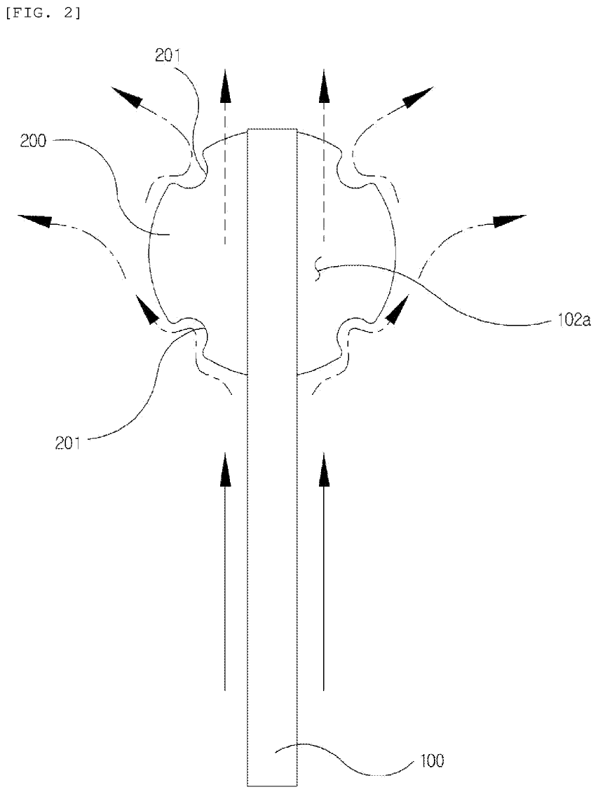

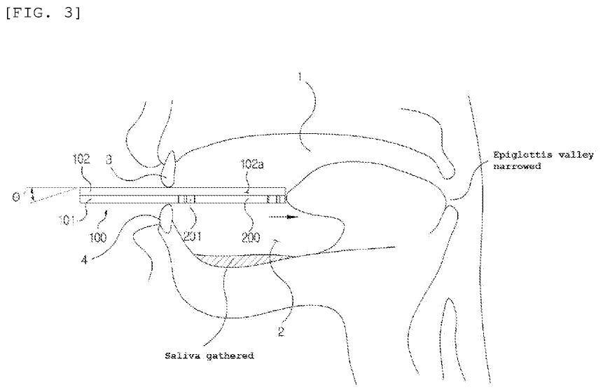

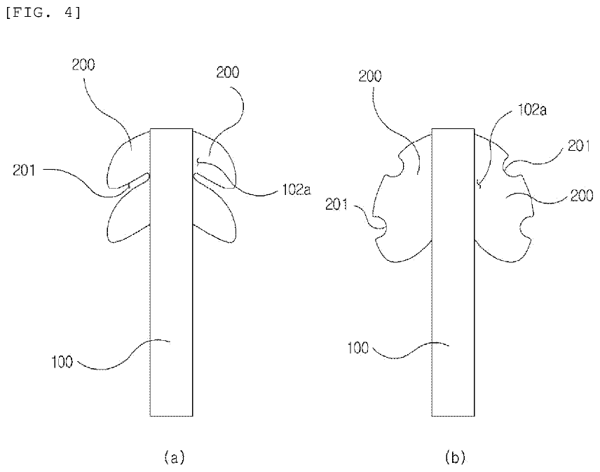

Referring to FIGS. 1 to 4, the horizontal bar 100 is formed in a non-penetrating bar shape, and may press the end of the tongue 1 such that the tongue 1 enters the oral cavity 2, wherein top and bottom surfaces of the bar are held between upper teeth 3 and lower teeth 4 and exposed out of the oral cavity 2, and air introduced into the oral cavity 2 through a gap between the upper teeth 3 and the lower teeth 4 is guided to flow in a horizontal direction.

The horizontal bar 100 is formed in a non-penetrating bar shape and air striking the horizontal bar 100 is dispersed to both sides with reference to the horizontal bar 100.

With respect to the horizontal bar 100, the lower jaw may be positioned below the horizontal bar 100 to prevent the secretion generated in the oral cavity 2 from flowing in the respiratory tract direction but to lead the same to be gathered in the oral cavity 2, wherein the secretion gathered in the oral cavity 2 goes down the throat hole along with saliva by swallowing the saliva.

The horizontal bar 100 may be made of silicone or spongy materials to protect the teeth.

The horizontal bar 100 preferably has a cross-section in a rectangular shape, but may also be formed in a polygonal shape if desired by a user.

The horizontal bar 100 may have one or more grooves or non-slip surfaces formed on a top surface and a bottom surface so that the teeth engaged therewith are not slippery depending on kinds of the material.

The horizontal bar 100 may consist of a lower bar 101 having the wing part 200 formed at the end thereof, and an upper bar 102 which protrudes from a top surface of the lower bar 101 in a bar shape and has a thru-hole 102a formed on both sides at the end of the upper bar in order to pass air introduced into the oral cavity 2 in a horizontal direction.

The lower bar 101 contacts the lower teeth 4 and the upper bar 102 contacts the upper teeth 3.

The thru-hole 102a may directly pass the air horizontally moving along the upper bar 102 and induce the air to strike the surface of the tongue 1.

The thru-hole 102a may enable breathing even if any site of the horizontal bar 100 is held between the upper teeth 3 and the lower teeth 4, thereby improving safety of a user and convenience in use.

The wing part 200 may protrude from the end of the horizontal bar 100 to both sides thereof and may be configured to reinforce inter-durability so as to prevent bending or twisting when entering the oral cavity 2.

The wing part 200 is guided by the horizontal bar 100 to lead the air introduced into the oral cavity so as to be diverted toward a wall surface of the oral cavity 2 and flow into the respiratory tract.

The wing part 200 may delay the air flow entering the respiratory tract so that a temperature of the air is changed to correspond to an internal temperature of the oral cavity 2, while allowing the air to contain moisture.

In this case, a temperature of respiratory air at room temperature of about 25° C. may be elevated and maintained at an average of about 32° C., and a surface temperature of the oral cavity is gradually decreased from the body temperature to about 34° C. to thus relieve the fever.

Preferably, the wing part 200 is formed to be close to the teeth holding the horizontal bar, whereby temperature change and moisture evaporation occur from the front side of the oral cavity 2.

The wing part 200 may have the same thickness as the lower bar 101.

The wing part 200 may be formed at a predetermined distance apart from the wall surface of the oral cavity 2, so as not to interfere with the wall surface of the oral cavity 2.

The wing part 200 may be formed in any one of semicircular, semi-elliptic and polygonal shapes.

The wing part 200 may further include at least one passage groove 201 on an outer surface thereof.

The passage groove 201 allows entry of external air flowing along the outer surface of the wing part 200 to thus delay the air flow moving toward the wall surface of the oral cavity 2, thereby improving treatment efficiency.

The cough treatment device according to the present invention configured as described above will be used as follows.

First, while maintaining a correct posture, the horizontal bar 100 is held and moved such that the end of the horizontal bar 100 is brought into contact with the end of the tongue, and then, the end of the tongue 1 is pressed such that the tongue 1 is pressed into the oral cavity 2 to thus modify a shape of the respiratory tract, thereby extending the respiratory tract (S100).

Then, top and bottom surfaces of the horizontal bar 100 pressing the end of the tongue 1 are held between upper teeth 3 and lower teeth 4 in order to fix the horizontal bar 100 between the upper teeth 3 and the lower teeth 4, whereby the wing part 200 provided at the end of the horizontal bar 100 is positioned in the oral cavity 2 (S200).

Thereafter, when a wearer breathes, external air flows into the oral cavity 2 through a gap between the upper teeth 3 and the lower teeth 4 of the wearer. In this case, the external air flows along the horizontal bar 100, is guided to the wing part 200 and is changed to correspond to an internal temperature of the oral cavity 2 while being diverted toward a wall surface of the oral cavity 2. At the same time, the air containing moisture is introduced into the respiratory tract (S300).

Herein, when the tongue 1 is pressed backward by the horizontal bar 100, a space in the epiglottis valley, as a main cause of the respiratory tract obstruction, may be narrowed to thus prevent the secretion from being caught or gathered and then overflowing. Further, an angle of the lower jaw opened by the horizontal bar 100 as well as the backward pressing of the tongue 1 cause the secretion generated in the oral cavity 2 to be gathered and isolated in the oral cavity 2 without flowing in the respiratory tract direction.

Further, the horizontal bar 100 may guide external airflow to be dispersed toward both sides of the upper bar 102 and the lower bar 101. At this time, the external air moving along the lower bar 101 undergoes a change in temperature while being diverted toward the wall surface of the oral cavity 2, and the air containing moisture is introduced into the respiratory tract.

More specifically, since the wing part 200 has at least one passage groove 201 on an outer surface thereof, external air flowing along the outer surface of the wing part 200 enters the passage groove 201 so that the air flow moving toward the wall surface side of the oral cavity 20 is further delayed and therefore the external air is changed to correspond to an internal temperature of the oral cavity 2 while containing moisture, simultaneously.

In addition, the external air moving along the upper bar 102 directly passes through the thru hole 102a to strike the surface of the tongue 1, and then, spreads in both side directions of the tongue 1 and upward. As a result, the temperature is changed and the air containing moisture is introduced into the respiratory tract.

That is, the external air guided to the upper bar 102 and the lower bar 101 of the horizontal bar 100 and introduced into the respiratory tract may reduce heat in a fever site without irritation such as dried pharynx, change in temperature, high friction of air, etc., but is not too much chilled, whereby respiratory tract swelling and narrowing may be relieved while facilitating recovery from inflammation. As a result, coughing may be naturally sedated.

Herein, the cough treatment device of the present invention is preferably disposed in the oral cavity 2 and a cough treatment time may be 1 minute to 10 minutes.

Hereinabove, an embodiment of cough treatment has been described wherein the treatment is implemented by breathing while holding and fixing the horizontal bar 100 between teeth to locate the wing part 200 in the oral cavity 2.

However, if some wearers suffer from coughing during breathing while holding and fixing the horizontal bar 100 between teeth, cough treatment may proceed by, after removing the wing part 200 from the oral cavity 2 and coughing, inserting the wing part 200 again into the oral cavity 2 according to the same procedure as described above.

In this case, the cough treatment device may be used after being covered with a sanitary cap (not shown) to enclose the horizontal bar 100 and the wing part 200. Thereafter, the device may also be repeatedly used while covering the same with other sanitary caps (not shown), and the sanitary cap is preferably disposable.

Further, in order to repeatedly use the cough treatment device, the cough treatment device may be portably used while being accommodated in a necklace type storage case (not shown) to enclose the wing part 200 as well as the horizontal bar 100.

As described above, a structure in which the horizontal bar 100 is held and fixed between the upper teeth 3 and the lower teeth 4 to dispose the wing part 200 in the oral cavity 2, wherein the wing part 200 guides air to pass toward a wall surface of the oral cavity 2, may have an advantage in that the tongue 1 pressed at the end of the horizontal bar 100 enters the oral cavity 2 to modify a shape of the respiratory tract and extend the respiratory tract, whereby air can be smoothly supplied to the extended respiratory tract to allow active breathing, thereby efficiently reducing heat around the inflammation site. In particular, when the tongue 1 is pressed backward by the horizontal bar 100, a space in the epiglottis valley, as a main cause of the respiratory tract obstruction, may be narrowed to thus prevent the secretion from being caught or gathered and then overflowing. Further, an angle of the lower jaw opened by the horizontal bar 100 as well as the backward pressing of the tongue 1 may cause the secretion generated in the oral cavity 2 to be gathered and isolated in the oral cavity 2 without flowing in the respiratory tract direction, thereby relieving the coughing caused by the secretion. In addition, when the air supplied to the oral cavity 2 through a gap between the upper teeth 3 and the lower teeth 4 is diverted toward the wall surface of the oral cavity 2 by the wing part 200, cough-inducing factors such as dried pharynx due to air, a change in temperature, air friction, etc. may be overcome to thus maintain stable breathing. Further, a temperature of respiratory air at room temperature of about 25° C. is elevated and maintained at an average of about 32° C., and a surface temperature of the oral cavity 2 is gradually decreased from the body temperature to about 34° C., and the air rubs against a heated site such as the tonsil or larynx to relieve fever, whereby cough symptoms may be alleviated to thus treat respiratory infection.

As described above, the cough treatment device according to the present invention is not limited to the embodiments described above and may include technical spirits in a range of various alterations and modifications implemented by those skilled the art to which the present invention pertains, without departing from the scope of the present invention.

1. A cough treatment device, comprising:
a horizontal bar which is formed in a non-penetrating bar shape and presses the end of a tongue such that the tongue centers an oral cavity, wherein top and bottom surfaces of the bar are held between upper teeth and lower teeth and are exposed out of the oral cavity, and air introduced into the oral cavity through a gap between the upper teeth and the lower teeth is guided to flow in a horizontal direction; and
a wing part which protrudes from opposite sides of the horizontal bar at the end thereof and induces the air guided by the horizontal bar and introduced into the oral cavity to be diverted toward a wall surface of the oral cavity and flow into the respiratory tract.

2. The device according to claim 1, wherein the horizontal bar comprises:
a lower bar having the wing part formed at the end thereof; and
an upper bar which protrudes from a top surface of the lower bar in a bar shape and has a thru-hole formed on both sides at the end of the upper bar in order to pass air introduced into the oral cavity in a horizontal direction.

3. The device according to claim 1, wherein the wing part is formed in any one of semicircular, semi-elliptic and polygonal shapes.

4. The device according to claim 1, wherein the wing part further includes at least one passage groove formed on an outer surface thereof.

5. The device according to claim 3, wherein the wing part further includes at least one passage groove formed on an outer surface thereof.
